(19) United States
(12) Patent Application Publication
(10) Pub. No.: US 2012/0288523 A1
(43) Pub. Date: Nov. 15, 2012

(54) ADMINISTRATION ROUTE FOR A COMPOSITION TO PROTECT AN ANIMAL AGAINST RHODOCOCCUS EQUI

(76) Inventor: Antonius Arnoldus Christiaan Jacobs, Boxmeer (NL)

(21) Appl. No.: 13/519,011

(22) PCT Filed: Jan. 3, 2011
(86) PCT No.: PCT/EP2011/050010

§ 371 Date: Jun. 25, 2012

Related U.S. Application Data
(60) Provisional application No. 61/292,055, filed on Jan. 4, 2010.

(30) Foreign Application Priority Data
Jan. 4, 2010 (EP) 10150042.9

(51) Int. Cl. A61K 39/02; C12N 1/20; A61P 31/04

(52) U.S. Cl. 424/234.1; 435/252.1

(57) ABSTRACT

The present invention pertains to the use of Rhodococcus equi antigens to manufacture a composition for administration to an animal, to elicit in the animal an immune response directed against Rhodococcus equi bacteria, which composition is in a form suitable for rectal administration.

The current invention pertains to a new administration route for a composition that protects an animal (the term “animal” includes humans), against an infection with Rhodococcus equi bacteria. Rhodococcus equi (formerly known as Corynebacterium) may cause pneumonia in foals and is also connected to opportunistic infections in immunocompromised subjects, in particular AIDS patients.

Compositions to protect a subject animal against a disorder arising from an infection with Rhodococcus equi bacteria have been made available over the past years. Different routes of administration have been described for these compositions such as intratracheal administration, parenteral administration and oral administration. Intratracheal administration may provide good results but is disadvantageous since it is relatively cumbersome to administer a vaccine directly into the trachea. Parenteral administration often gives rise to severe abscesses and is therefore not preferred, in particular when humans or horses are to be protected against a Rhodococcus equi infection. Oral administration therefore has since decades been the preferred route of administration (e.g. J. M. Chirino-Trejo et al; Canadian Journal of Veterinary Research, 1987, 51(4), pp. 444-447), but gives rise to varying success in levels of protection.

It is an object of the present invention to find a convenient administration route for a composition that elicits in an animal an immune response directed against Rhodococcus equi bacteria, which immune response develops fast and gives rise to adequate protection against an infection with virulent Rhodococcus equi bacteria. Rectal administration has been found to meet this object. Surprisingly, rectal administration of such a composition, when compared with oral administration, gives rise to very fast development of an immune response and also, leads to adequate protection. More importantly, rectal vaccination has proven to lead to no, or at least no significant, variation in the induced immune response. This is a very important improvement when compared to prior art administration methods, in particular when compared to the widely used oral administration method. The reason for the difference in immune response after oral or rectal administration is not clear. Based on prior art knowledge, one would expect a comparable immune response when assessing oral and rectal vaccination against Rhodococcus equi. In any case, through the fast induction of an adequate immune response that leads to interference with the pathogenic micro-organism in the host animal, the infection or the clinical signs of the resulting disease will at least be diminished, or can even be prevented completely. Also the further spread of the disease in the environment may be halted or diminished.

The current invention can be used to manufacture a composition for administration to an animal, to elicit in the animal an immune response directed against Rhodococcus equi bacteria. The required Rhodococcus equi antigens may be any antigenic material derived directly from Rhodococcus equi bacteria, or antigenic material artificially made (such as recombinant antigens or antigens made by physico-chemical production methods). Such antigens may for example be bacterial subunits such as proteins or polysaccharides derived from the outer surfaces of the bacterial cell (capsular antigens) or from the cell interior (the somatic or O antigens). They may also be excretion products, killed (whole) bacteria or live, preferably attenuated, bacteria. Attenuated bacteria are incapable of inducing a full suite of symptoms of the disease that is normally associated with its virulent (often wild-type) pathogenic counterpart. Attenuated bacteria exhibit a reduced ability to survive in a host, and may contain one or more mutations in one or more virulence genes.

In order for the invention to be applied, the composition should be in a form suitable for rectal administration. Such a form could be a liquid (for example based on water or another pharmacologically acceptable fluid or mixture of fluids), a paste (such as known for the administration of anti-parasitic molecules such as ivermectin), a gel, a suppository, a spray (e.g. administered via a cannula), a fast-disintegrating tablet (such as known from U.S. Pat. No. 5,384,124 assigned to Farmalyoc) or any other suitable form.

In general, a composition for use in the present invention can be manufactured by using art-known methods that basically comprise admixing the antigens (or a constitution containing the antigens) with a pharmaceutically acceptable carrier. Optionally other substances such as adjuvants, stabilisers, viscosity modifiers or other components (such as antigens from other micro-organisms, in particular Salmonella spp, Escherichia spp, Lawsonia spp or other pathogens that infect the gastro-intestinal tract) are added depending on the intended use or required properties of the vaccine.

For application of the present invention it is preferred that the antigens comprise live Rhodococcus equi bacteria. In an embodiment the composition comprises at least between 1×10⁷ and 1×10¹⁰ CFU (colony forming units) live attenuated Rhodococcus equi bacteria per dose, preferably between 1×10⁹ and 1×10¹⁰ CFU. Preferably, the composition elicits an adequate immune response after prime administration only.

The present invention is suitable for treating unweaned foals. This is a group of animals which is very susceptible for a Rhodococcus equi infection. Many infected animals die, whereas survivors appear to have lost lung capacity permanently

The present invention is also directed to a composition comprising Rhodococcus equi antigens for administration to an animal, to elicit in the animal an immune response directed against Rhodococcus equi bacteria, which composition is in a form suitable for rectal administration. The invention is also directed to a method for protecting an animal against a disorder arising from an infection with Rhodococcus equi bacteria, comprising rectal administration of a composition containing Rhodococcus equi antigens. It is noted that “protection” in the sense of the present invention means aiding in preventing, ameliorating or curing a disease or disorder, which in connection with the present invention is a disease or disorder associated with an infection with virulent Rhodococcus equi bacteria.

The invention will be illustrated based on the following examples.

Example 1 describes several compositions that upon administration to a subject animal elicit an immune response directed against Rhodococcus equi bacteria.

Example 2 shows that high antibody titres correspond to protection against an infection with Rhodococcus equi bacteria.

Example 3 shows that rectal administration provides significantly better results than oral administration.

Example 4 shows additional results for rectal vaccination.

### EXAMPLE 1

Compositions for eliciting an immune response against Rhodococcus equi bacteria are generally known in the art. In European patent application 09150379.7, filed 12 Jan. 2009, assigned to Intervet International BV, three different mutant bacterial strains are described (in particular see section “B1 used strains” under “Strains according to the invention”: Strain RE1ΔipdAB, strain RE1ΔipdAB-AD+ and strain RE1ΔipdABipdAB2. With each of these strains a composition to elicit an immune response against Rhodococcus equi bacteria is made by mixing these strains with a pharmaceutically acceptable carrier. Such a carrier may for example be Nobivac diluent (available from Intervet, Boxmeer, The Netherlands), alginate gel or a Pronutrin suspension (wherein Pronutrin itself is available in the form of granules from Boehringer Ingelheim, Germany).

This way, compositions were made comprising the strains RE1ΔipdAB and RE1ΔipdABipdAB2 in Nobivac diluent (type “A”), alginate gel (type “B”) or a Pronutrin suspension (type “C”).

Compostions of type A were made by mixing live bacteria with the diluent.

Compositions of type B were made by mixing 9 ml of a Composition of type A, with 66 ml alginate gel (5% w/w Sodiumalginate in 0.04 M phosphate buffered saline) to arrive at 75 ml alginate gel mixture.

Compositions of type C were made by first mixing 14 grams of Pronutrin with 42.5 ml of water (Mixture A). Next to this, Mixture B was made, consisting of 1.5 ml physiological salt (0.9% NaCl) and 6 ml of a Composition of type A. Just before administration, Mixture A and B were mixed, and 4 grams of Horsefood “Electrolyten Mix” (available from Van Gorp Diervoeders, Waalwijk, The Netherlands) were added. This resulted in a Composition of type C.

### EXAMPLE 2

### Experimental Design

Fourty six foals were used for the experiment. Eleven foals (Group 1) were vaccinated twice orally (2-week interval) with 1 ml of Composition A, containing 5×10⁷-5×10⁹ bacteria of strain RE1ΔipdAB per dose (for four foals the dose was of 5×10⁷ CFU, for three other foals the dose was 5×10⁹, and for the remaining four foals the dose was 5×10⁹ CFU). Eight foals were vaccinated twice orally (2-week interval) with 1 ml of Composition A, containing 1×10⁷ bacteria of strain RE1ΔipdABipdAB2 per dose (Group 2). Eight foals were vaccinated once orally with 1 ml of Composition A containing the same dose (Group 3). Three foals were vaccinated once gastrically with 100 ml of Composition A containing 1×10¹⁰ bacteria of strain RE1ΔipdAB per dose (Group 4).

Sixteen foals were left unvaccinated as controls. Two weeks after repeated dose vaccination or three weeks after single dose vaccination all fourty six foals were challenged intratracheally with a virulent Rhodococcus equi strain by administering 100 ml PBS containing 4×10⁴ CFU bacteria per ml. During a period of 3 weeks after challenge the foals were clinically evaluated.

The foals were weighed at day of first vaccination, day of challenge and at day of necropsy. Serum blood was sampled at day of each vaccination, day of challenge and at day of necropsy, to determine antibody titres. At 3 weeks after challenge (or earlier in case of severe clinical signs) the foals were weighed and euthanized and a complete post-mortem examination was performed with special attention to the lungs and respiratory lymph nodes. The lungs were weighed in order to calculate the lung to body weight ratio. Tissue samples from all lung lobes were sampled for bacteriological examination and counting. Additional samples for bacteriology and histology were collected from all abnormalities encountered during necropsy.

### Results and Discussion

Lung scores were established as described in connection with Table 7 in European patent application 09150379.7. Sera at day of challenge were tested for antibodies directed against Rhodococcus equi bacteria in an ELISA test. The controls were all low to negative with respect to antibody titres.

The noted effects were as followed. In Group 1, five out of eleven foals were protected against the challenge with virulent Rhodococcus equi (they had a lung score below 50). Six foals were not protected, they developed mild to severe pneumonia (lung score between about 100 and 350). There was no correlation between dose and response in this group. In the Groups 2 and 3 combined, there were four foals protected, two foals in Group 2 and two foals in Group 3. Twelve foals of these two groups developed pneumonia. All three foals in Group 4 appeared to be protected.

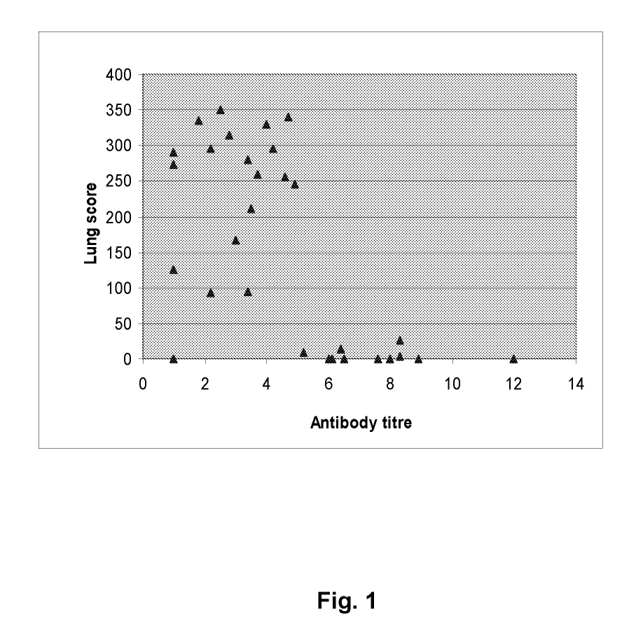

In FIG. 1 the correlation between serum titre and lung score is given for the vaccinated animals. It appears that there is a 100% correlation between a serum antibody titre above 5 (on a 2 log scale, i.e. 2-fold serial dilutions were made) and protection against a challenge with virulent Rhodococcus equi bacteria after gastro-intestinal vaccination. This correlation does not necessarily mean that the antibodies provide the protective effect. It may be that the protective effect is provided by cellular immunity and at the same time, that the antibody titre (after gastro-intestinal administration of the antigens) corresponds to the level of induced cellular immunity.

The low number of protected foals, as well as the absence of a dose-response cannot be explained based on the experimental set-up and the results obtained. It could be related to dose, dose-volume, vaccination method or scheme, the pH of the stomach, other unknown factors (such as breed of the foals) or a combination of these. What has been unambiguously established though, is that a serum titre above 5, for gastro-intestinal vaccination, correlates to adequate protection against a challenge with virulent Rhodococcus equi.

### EXAMPLE 3

In this example a comparison between oral and rectal vaccination of several different compositions containing Rhodococcus euqi antigens is described. For this, the Compositions A, B and C as mentioned in Example 1 were used, each containing Rhodococcus equi strain RE1ΔipdABipdAB2.

Composition A was used for rectal vaccination. Each dose contained 3 ml of the composition (corresponding to a bacterial dose of 10¹⁰ CFU). Composition B was used for oral vaccination. Each dose consisted of 25 ml of the gel mixture (corresponding to a bacterial dose of 10¹⁰ CFU). Composition C was also used for oral vaccination. Each dose of this Composition C consisted of 25 ml of the Pronutrin suspension (corresponding to a bacterial dose of 10¹⁰ CFU).

A first group of two foals were rectally vaccinated, solely by prime vaccination with Composition A (Group 1). A second group of three foals were rectally vaccinated by prime and boost vaccination (2 weeks apart) with Composition A (Group 2). A next group of three foals were orally vaccinated by prime and boost vaccination (2 weeks apart) with Composition B (Group 3). Lastly, a group of two foals were orally vaccinated by prime and boost vaccination (2 weeks apart) with Composition C. The titres against Rhodococcus equi bacteria were measured in line with Example 2. The (mean) results are indicated in the table below.

TABLE 1
Mean serum titre against Rhodococcus equi
after several vaccination regimes

| | Days after prime vaccination | | | | |
|---|---|---|---|---|---|
| Group No | 0 | 14 | 21 | 28 | 35 |
| Group 1 | 2.5 | 8.3 | 9.2 | 8.1 | 7.6 |
| Group 2 | 2.4 | 7.0 | 7.2 | 7.5 | 7.1 |
| Group 3 | 4.2 | 3.6 | 5.7 | 6.5 | 5.8 |
| Group 4 | 2.8 | 2.6 | 2.8 | 3.0 | 3.8 |

Surprisingly, all foals that were rectally vaccinated, whether or not receiving solely prime vaccination or additional boost vaccination, developed titres above 5.0 very quickly (in less than 14 days after receiving the first vaccination). The foals that received the alginate gel also developed protective titres, but this took about three weeks. The last group did not develop protective titres. There is no clear explanation for this, but it may explain why oral vaccination against Rhodococcus equi in the prior art has regularly provided non-satisfactory results. Given the fact that the only essential difference between the four groups is the route of administration, it may be concluded that for any composition containing Rhodococcus equi antigens (i.e. antigens that induce an immune response directed against Rhodococcus equi bacteria), rectal vaccination will provide a very adequate immunological response in the vaccinated subject.

### EXAMPLE 4

In this example the experiment of Example 3 was repeated to see whether or not the results are consistent, to see whether or not a ten times lower dose would also provide an adequate result and to see in more detail how the antibody titre develops during the first fourteen days after vaccination.

The same vaccine and test set-up as described in Example 3 was used. Again, composition A was used for rectal vaccination. A first group of three foals were rectally vaccinated, solely by a single prime vaccination with Composition A at day 0 with a dose of 10¹⁰ CFU (Group 1). A second group of three foals were rectally vaccinated by a double prime vaccination with Composition A at day 0 and 1, each vaccination at a dose of 10¹⁰ CFU (Group 2). A next group of three foals were rectally vaccinated solely by a single prime vaccination with Composition A at day 0 with a dose of 10⁹ CFU. (Group 3).

The titres against Rhodococcus equi bacteria were measured in line with Example 2. The (mean) results are indicated in the table below.

TABLE 1
Mean serum titre against Rhodococcus equi
after several vaccination regimes

| | Days after prime vaccination | | | | |
|---|---|---|---|---|---|
| Group No | 0 | 7 | 10 | 14 | 21 |
| Group 1 | 1.5 | 1.5 | 4.4 | 6.5 | 6.4 |
| Group 2 | 2.0 | 6.6 | 9.7 | 9.7 | 10.1 |
| Group 3 | 2.6 | 2.4 | 5.7 | 7.3 | 6.5 |

It can be seen that in all groups, protective titers are obtained within 14 days, or even within 10 days. This is extremely fast and unambiguous (i.e. no significant variation in immune response, all foals had protective titers at 14 days after vaccination) when compared to oral vaccination. In group 2, which received a double prime vaccination, protective titers were even obtained within 7 days.

1. (canceled)

2. The method according to claim 1, characterised in that the antigens comprise live Rhodococcus equi bacteria.

3. The method according to claim 2,
characterised in that the composition comprises at least between 1×10⁷ and 1×10¹⁰ CFU live attenuated bacteria per dose.

4. The method according to claim 3, characterised in that the composition comprises between 1×10⁹ and 1×10¹⁰ CFU bacteria per dose.

5. The method according to claim 3, characterised in that the composition elicits the immune response after prime administration.

6. The method according to claim 5, characterised in that the composition is for administration to unweaned foals.

7. A composition comprising Rhodococcus equi antigens for rectal administration to an animal, to elicit in the animal an immune response directed against Rhodococcus equi bacteria for protecting an animal against a disorder arising from an infection with Rhodococcus equi bacteria.

8. A method for protecting an animal against a disorder arising from an infection with Rhodococcus equi bacteria, comprising rectal administration of a composition containing Rhodococcus equi antigens.

9. The method according to claim 3, characterised in that the composition elicits the immune response after prime administration.

10. The method according to claim 2, characterised in that the composition elicits the immune response after prime administration.

11. The method according to claim 1, characterised in that the composition elicits the immune response after prime administration.

12. The method according to claim 11 characterised in that the composition is for administration to unweaned foals.

13. The method according to claim 10 characterised in that the composition is for administration to unweaned foals.

14. The method according to claim 9 characterised in that the composition is for administration to unweaned foals.

15. The method according to claim 4 characterised in that the composition is for administration to unweaned foals.

16. The method according to claim 3 characterised in that the composition is for administration to unweaned foals.

17. The method according to claim 2 characterised in that the composition is for administration to unweaned foals.

18. The method according to claim 1 characterised in that the composition is for administration to unweaned foals.
